(19) United States
(12) Patent Application Publication
(10) Pub. No.: US 2008/0234563 A1
(43) Pub. Date: Sep. 25, 2008

(54) Device for and Method of Delivery and Removal of Substances in and From a Tissue or Vessel

(75) Inventor: Werner Regittnig, Graz (AT)

Correspondence Address: SMITH FROHWEIN TEMPEL GREENLEE BLAHA, LLC, Two Ravinia Drive, Suite 700, ATLANTA, GA, 30346, US

(73) Assignee: Medizinishe Universitat Graz, Graz (AT)

(21) Appl. No.: 11/994,739

(22) PCT Filed: Jul. 5, 2006
(86) PCT No.: PCT/EP2006/006568

§ 371 Date: Jun. 2, 2008

(30) Foreign Application Priority Data
Jul. 6, 2005 (AT) A 1140/2005

(51) Int. Cl. A61B 5/157; A61B 5/00

(52) U.S. Cl. 600/365; 600/578

(57) ABSTRACT

The invention relates to a device (5) for delivery of substances (9) to and removal of bodily substances (22) from a tissue (2) or vessel (25) of a body using an element (6) to be positioned in the tissue (2) or vessel (25). To provide a device of this type for the delivery and removal of substances (9, 22), which has a small and robust construction and may be used as easily and comfortably as possible by the patient, the element (6) is at least partially made of porous material and comprises at least one inlet (7) for supplying the substance (9) to be delivered and at least one outlet (10) for draining the bodily substances (22), the inlet (7) being connectable via an inlet line (13) to a container for the substances (9) to be delivered, so that in the event of a pressure gradient between inlet (7) and outlet (10), the substance (9) to be delivered, during the traversal of the porous element (6), is delivered via the mantle surface of the porous element (6) to the tissue (2) or vessel (25) and the bodily substances (22) are extracted from the tissue (2) or vessel (25) via the mantle surface of the porous element (6) and drained via a drain line (12) connectable to the outlet (10).

This application claims the benefit of the filing date of the Austrian Patent Application No. 2B A 1140/2005-1 filed Jul. 6, 2005, the disclosure of which is hereby incorporated herein by reference.

The invention relates to a device for delivery of substances to and removal of bodily substances from a tissue or vessel of a body with an element to be positioned in the tissue or vessel.

Furthermore, the invention relates to a system and method for delivery of medications to a tissue or vessel depending on the concentration of a specific bodily substance in the tissue or vessel.

In the treatment and diagnosis of illnesses, the necessity very frequently exists of repeatedly removing bodily fluids and repeatedly introducing medications or therapeutic fluids into specific internal regions of the body. An example of this is the current standard treatment of type 1 diabetes, in which the fingertip blood for a glucose determination is removed repeatedly (two to ten times daily) and insulin is administered into the subcutaneous adipose tissue repeatedly (two to five times daily).

In order to reduce the number of needle sticks required and the pain and unpleasantness connected therewith during repeated introductions and removals of substances, different types of catheters have been developed which may be implanted in a bodily region and then allow access to this bodily region over a longer period of time. Depending on the type of the implanted indwelling catheter, this access provided may be used for delivery of substances to this bodily region and/or for removal of bodily substances from this bodily region. An example of this would be the transcutaneous indwelling catheter, via which patients having type 1 diabetes may be supplied with insulin continuously into the subcutaneous adipose tissue. Typical transcutaneous indwelling catheters are composed of stiff hollow needles or soft cannulas, which may be inserted by the patient into the subcutaneous tissue of the abdomen, fixed with an adhesive strip, and connected to an insulin pump using tubing.

For example, U.S. Pat. No. 5,257,980 describes a flexible, transcutaneous cannula, which is introduced by the patient into the subcutaneous tissue with the aid of a metal needle and then may dwell for a long time in the tissue after removal of the metal needle. Transcutaneous indwelling catheters of this type may only be used for delivery of substances to tissue. Removal of substances from tissue, for example glucose for the purpose of determining the tissue glucose concentration, is not possible using these indwelling catheters.

Examples of indwelling catheters which are suitable both for the delivery and also for the removal of substances would be types of catheters which operate according to the principles of microdialysis, ultrafiltration, or microperfusion.

For example, U.S. Pat. No. 5,741,284 describes an indwelling catheter operating according to the principle of microdialysis. This catheter has a membrane positioned at the end of the catheter and is introduced into the tissue using a hollow needle enveloping the membrane. After removal of the hollow needle, the catheter is perfused with an isotonic fluid. In the event of concentration differences between the materials dissolved in the perfusate fluid and the materials dissolved in the tissue fluid, there is a diffusion-driven transport of substances through the catheter membrane and thus an exchange of substances between perfusate fluid and tissue fluid. Depending on the direction of the concentration gradient, materials from the tissue fluid may thus be brought via the membrane into the perfusate fluid or perfusate fluid materials may be brought via the membrane into the tissue. Microdialysis catheters have a very low mechanical strength because of the thin membrane, due to which the introduction and removal of the catheter has been shown to be difficult and complex. In addition, the manufacturing of microdialysis catheters of this type is relatively costly.

An indwelling catheter operating according to the principle of ultrafiltration is known, for example, from U.S. Pat. No. 5,002,054. This catheter comprises multiple hollow fibers positioned at the end of the catheter and is introduced into the tissue using a hollow needle enveloping the fibers. After the removal of the hollow needle, a convection of materials through the porous fiber walls is caused because of a pressure difference generated between tissue fluid and catheter fluid. Depending on the direction of the pressure gradient, tissue fluid and materials dissolved therein may thus be suctioned via the fiber wall into the fiber cavity or fluids and materials dissolved therein from the fiber cavity may be introduced via the fiber wall into the tissue. Ultrafiltration catheters have a very low mechanical strength because of the thin fiber walls, due to which also the introduction and removal of the catheters have been shown to be difficult and complex. In addition, the manufacturing of ultrafiltration catheters of this type is also costly.

U.S. Pat. No. 6,706,009 B2 describes an indwelling catheter which operates according to the principle of open microperfusion. This catheter comprises a cannula provided with macroscopic perforations and is introduced into the tissue using a metal needle positioned in the cavity of the cannula. After removal of the metal needle, the perforated cannula is perfused with an isotonic fluid. In the event of concentration differences between the materials dissolved in the perfusate fluid and the materials dissolved in the tissue fluid, there is a diffusion-driven transport of substances through the perforated cannula wall. Depending on the direction of the concentration gradient, materials from the tissue fluid may thus also be brought via the perforated cannula wall into the perfusate fluid here or, vice versa, perfusate fluid materials may be brought via the perforated cannula wall into the tissue. Microperfusion catheters of this type are more stable than microdialysis or ultrafiltration catheters because the thin membrane is dispensed with, but their exchange of substances is less efficient in comparison thereto. In order to achieve an efficiency in substance exchange which is comparable to microdialysis and ultrafiltration catheters, microperfusion catheters may be equipped with an increased number of perforations. This increase in efficiency is at the cost of significantly larger constructions and therefore more difficult placement of the catheters.

Diabetes is one of the most frequent chronic illnesses worldwide. Diabetes is characterized by the loss of insulin production of the pancreas (type 1 diabetes) or by an abnormal insulin secretion of the pancreas (type 2 diabetes). In order to avoid states of high blood glucose concentration (hyperglycemia) and their acute life-threatening consequences (ketoacidosis) and also chronic consequences (e.g.: blindness, amputation, kidney failure), patients having type 1 diabetes must be externally supplied with insulin. Patients having type 2 diabetes must also be supplied with insulin if a reduction of the blood glucose level may not be achieved by diet and by giving oral antidiabetic agents.

Insulin is introduced by a majority of type 1 diabetic patients multiple times a day into the adipose tissue located under the skin (subcutaneous insulin injection) using an injection needle. An increasing number of type 1 diabetic patients are using an insulin pump and a transcutaneous indwelling catheter worn therewith for the continuous supply of insulin. This so-called insulin pump therapy represents the most effective form of therapy for type 1 diabetes at this time.

A large difficulty in insulin substitution is adapting the insulin dose to the existing blood glucose concentration. Thus, for example, if too high a dose is selected, too strong a glucose reduction and a life-threatening state (coma due to hypoglycemia) connected thereto may be provoked. Furthermore, the typical type of blood glucose measurement using a glucose measuring unit and blood removal by sticking the fingertips may not be performed frequently enough for practical reasons in order to recognize states of high glucose concentration in a timely manner. These and other difficulties in the current form of treatment therefore make it nearly impossible to achieve normoglycemic states over a long daily period of time. Therefore, in most cases, the current forms of treatment for type 1 diabetes may not prevent the development of the above-mentioned late complications.

U.S. Pat. No. 5,325,867 discloses a device for the sampling of body fluids using a hollow needle, and is provided with a storage system with several separate sample containers placed on a movable carrier. The containers which are sealed by puncturable membranes so as to be gas-tight, will receive collected fractions of the body fluid at given intervals, a device connected to a control unit being provided, by which the hollow needle is positioned over each sample container in turn and is inserted through the membrane into the sample container. The sampling device is configured as a two-channel cannula, one of whose channels is connected via a first connecting tube to the hollow needle used for insertion into the sample containers, while its other channel is connected via a second connecting tube to a pump operated by the control unit. In their unused state the individual sample containers are filled within an amount of gas or gas mixture whose volume at atmospheric pressure is smaller than that of the body fluid to be received.

EP 0,958,780 A1 discloses an implanted depot dispensing measured quantities of a medical substance like insulin, joined to the skin with small disks and equipped with an elastic self closing membrane, has a supply catheter and a suction catheter attached to its bottom. The supply catheter is used for the insertion of a medical substance, the suction catheter facilitates the removal of body fluids for diagnostic purposes. A sensor can be attached to the suction tube in order to allow a check of the body fluid already inside the body.

DE 31 12 762 A1 discloses a catheter set which permits continuous withdrawal of blood via a central access to a vessel and simultaneous infusion of solutions distally thereof, which is required in medicine, in particular for systems in which infusions are controlled via a computer in accordance with a current blood value, e.g. glucose. It comprises two channels which are to be tightly connected, and an axial catheter fixed to the channel by a stopper strip and a union nut. Infusion can be made through the axial catheter, an anticoagulant solution can be fed in between the catheter and the inner channel, the solution being mixed with the blood between the sleeve end of the outer and inner channels, and a mixture of blood and anticoagulant can be sucked in between the inner and outer channels.

US 2001/0047170 A1 discloses endoluminal methods and devices for the removal of anatomical structures such as vascular structures under endoscopic visualization of the process from within the anatomical structure or surgical region of interest. The device provides an elongated flexible endoscopic guide which may be passed through the lumen of an elongate vessel or structure, such as for example, the saphenous vein from an entry veinotomy to an exit veinotomy. The guide provides passageways for a variety of surgical devices: an endoscope for viewing the surgical regions of interest, a cautery device and other surgical tools for performing ligation and other surgical procedures, and phleboextractor for removing elongate structures.

A porous flexible drain can be deployed to provide pharmacological agents or collecting fluid at the surgical site.

It is an object of the present invention to provide an efficient delivery and removal of substances.

The object may be solved by the independent claims.

The term “substance” generally includes greatly varying fluids having materials dissolved therein or also greatly varying gases.

The term “bodily substances” generally includes all substances which occur in the surroundings of the element positioned in the tissue or vessel.

The term “porous material” may particularly denote any material comprising pores or vessels or holes, particularly in the dimension of nanometres, micrometres, or millimetres. Such pores may be three-dimensional structures in a solid body. A plurality of such interconnected pores formed in a solid body may (for instance statistically) form pathways in the material through which particularly fluidic samples may move or penetrate. For instance, a sintered body, soil, or a rock may comprise pores.

The porous element may be a network of interconnected pores through which fluids having materials dissolved therein can be conducted. At the surface of such a porous structure, a contact and an exchange between the perfusion fluid and the surrounding tissue or vessel fluid may be enabled.

The pore size and density may be configured in such a manner that, when forming a pressure difference between an inlet and an outlet, the fluidic flow may be maintained in the porous structure without “sucking” the surrounding tissue or vessel components (for instance cells having a size of 10 μm to 150 μm or capillaries having a length of 500 μm and a thickness of 7 μm), which might cause surface portions of the pores to be closed or blocked. This can happen with very large pores sizes which may therefore be less favourable. For instance, the pore size may be smaller than 200 μm, since with significantly larger pore sizes it is believed that cells and capillaries may be pressed into the pores. On the other hand, extremely small pores (less or significantly less than 0.1 μm) may have a hydraulic conductivity being much less than that of the surrounding tissue. The pressure difference required for the solution to penetrate the porous element might then be so large that the surrounding tissue or vessel structures may be harmed or damaged.

Porous elements may be manufactured by sintering polymers, ceramics or metals in granular shape. Also sintering metallic fibres may generate a porous structure.

A further efficient method for manufacturing porous plastic structures is expanding specific plastics (like expanded polytetrafluoroethylene (ePTFE) or expanded fluorinated ethylene propylene (eFeP)).

Another method for depositing porous layers on, for instance, non-porous surfaces is coating. In this respect, porous metal, porous ceramics, and porous polymer layers may be manufactured (for example titanium coating, ceramic coating, polymer coating).

In the context of this application, the term “porous material” may also include patterned rough surfaces (surface texturing). When such rough surfaces (including for example surface grooves, surface channels, surface pyramids) have appropriate dimensions (like in the order of magnitude of 1 μm), it may be possible that a liquid flow can occur along (on and/or in) such a rough surface without the surrounding tissue or vessel structures being drawn into the channels, grooves and/or surface valleys, thereby preventing that the flow is disturbed by such effects.

Methods that may be applied for patterning surfaces of metals, polymers, and/or ceramics are, for example, ion beam texturing, laser beam texturing, imprint lithography, microcontact printing, catalytic growth, or thin film coating.

According to an exemplary embodiment of the present invention, a device for delivery and removal of substances is provided which has a smaller and more robust construction than known indwelling catheters having comparable function and which may be used as easily and comfortably as possible by a patient. According to an exemplary embodiment of the present invention, it may be possible to combine the advantages of different known indwelling catheters and avoid and/or reduce their disadvantages. The device may also be suitable for a mass production at relatively low production costs and therefore also may be accessible to a broad circle of patients, for example, for the therapy of diabetes.

According to an exemplary embodiment of the present invention, a system is provided, which allows a proper simulation of the glucose-regulating function of a healthy pancreas over a long possible period of time and may thus be used for the therapy of type 1 diabetes. For high acceptance, the system may be constructed as small and compactly as possible and should be able to be worn comfortably on the body.

According to an exemplary embodiment of the present invention, a method is provided which allows a proper simulation of the glucose-regulating function of a healthy pancreas over a long period of time. In addition, the removal of glucose from a tissue or vessel and the delivery of insulin to the tissue or vessel is to be able to be performed efficiently by the method according to the invention.

This may be achieved in that a component of embodiments of the invention is at least partially formed by porous material. The porous material comprises multiple pores which are interconnected, so that fluids and materials dissolved therein may be conducted through the porous material. In order to implement the surface size and mechanical strength of the porous material in accordance with the requirements of application, the porous material may be implemented in multiple shapes and sizes. For example, the porous material may be designed as rod-shaped, tube-shaped, or plate-shaped. A system according to an exemplary embodiment of the invention also comprises an inlet line and a drain line that permit a fluid reservoir and a fluid collector to be placed in fluid communication with the porous material. The inlet line and drain line also allow a pressure differential, from a source external to the porous material, to be applied to surface regions of the porous material to promote controlled fluid movement through the porous material. Thus, embodiments of the invention do not rely solely on the intrinsic properties of the porous material to cause fluid flow, for example, by capillary action (i.e., wicking). The pressure differential can be created readily through use of a pumping system and applied to the porous material either in the form of positive pressure (so-called push mode), negative pressure (so called pull mode), or both (so-called push-pull mode). The device is placed into the selected tissue or vessel in such a way that the surface of the porous material is enclosed by the tissue or vessel, thereby allowing the porous material to be isolated or partitioned from the environment outside the tissue or vessel (e.g., air and atmospheric pressure). Based upon the creation of a pressure differential by the external pumping system, fluids and materials dissolved therein can then be drawn from the reservoir outside the body, into the inlet line, through the porous material, through the drain line, and into a collector outside the body. Because of concentration differences between the substances located in the porous material and the substances located in the tissue and/or because of pressure differences between porous material and tissue, an exchange of substances between the porous material and the surrounding tissue is caused during the flow through the porous material. Depending on the direction of the concentration and pressure gradients, substances may therefore be conveyed from the porous material into the tissue and/or substances may be conveyed from the tissue into the porous material. After flowing through the porous material, substances are moved from the porous material via the drain line connected to the outlet to a sensor and/or a collection container. Therefore, a substance, particularly a medication, such as insulin, may be delivered and also, preferably for the purpose of regulating the delivery of the substance, the concentration of specific bodily substances, such as the tissue glucose concentration, may be measured via one and the same implant, in that tissue fluid or blood plasma is removed and supplied to a sensor for determining the concentration of the materials dissolved therein. The device is distinguished by its special simplicity and is therefore producible very easily and cost-effectively. Finally, it is possible to manufacture the device having relatively small dimensions, which makes use easier for the patient. The device according to the invention combines the advantages of known microdialysis and ultrafiltration catheters with those of known microperfusion catheters, so that a more efficient substance exchange and a high degree of robustness result.

The element to be positioned in the tissue or vessel may be made at least partially of porous metal, porous ceramic material, or porous plastic, and should have biocompatible properties. An number of materials, such as polyethylene (PE) or polytetrafluorethylene (PTFE), are biocompatible and may be manufactured having a porous structure and in different shapes and sizes easily through sintering, for example.

The size of the pores of the porous material is, for example, 0.02 μm to 200 μm. The pore size is, of course, dependent on the substance to be delivered and/or the bodily substances to be extracted and has to be adapted to the particular conditions.

Special effects may be achieved if the element has pores of different sizes. Areas having pores and areas without pores may also be combined and therefore boundary layers made of porous and nonporous material may be produced.

The element may also have a porosity gradient, so that the pore size increases or decreases in a specific direction of the element. Advantages during delivery of substances and/or extraction of bodily substances may thus be achieved. If the element is produced through sintering, a porosity gradient of this type, according to which the pore size is changed depending on the geometry of the element, may be produced easily.

According to a further feature of the invention, the element is at least partially made of hydrophobic porous material. In particular, advantages for the delivery of substances may be achieved through a hydrophobic implementation of the surface.

It is also possible for the element to be at least partially made of hydrophilic porous material. A hydrophilic surface of the porous material may particularly be advantageous for the extraction of bodily substances from the tissue or vessel. In particular, advantages may be achieved through combinations of hydrophobic and hydrophilic surfaces of the porous material.

According to an embodiment variation of the invention, the element is formed by a rod made of the porous material, the inlet for connecting the inlet line for the connection to the container for the substance to be delivered being positioned at one end of the rod and the outlet for connecting the drain line for draining the extracted bodily substances being positioned at the other end of the rod, so that the delivery of the substance to the tissue or into the vessel and the extraction of the bodily substances from the tissue or vessel occurs via the mantle surface of the rod. This embodiment is distinguished by an especially simple production. Central element is the rod made of porous material, such as porous plastic, which may be produced from particles made of biocompatible polyethylene (PE) through sintering, for example. The inlet line for the substances to be delivered and the drain line for the removal of the bodily substances from the tissue or vessel may also be made of the base material of the element. The oblong element is preferably placed in the tissue or vessel with the aid of a hollow needle and the ends are connected to the corresponding lines. The substance to be delivered is pumped into the element made of porous material with the aid of a pump and partially delivered to the tissue or into the vessel. The substances extracted via the mantle surface of the porous rod are transported via the drain line to a sensor, for example, and, after determining the concentration of the substances, are transported further to a collection container. This embodiment variation has the disadvantage that the skin must be tunneled under and therefore must be penetrated twice to place the device.

A reinforcement element, around which the porous material is positioned, may be positioned in the interior of the rod for reinforcement.

The reinforcement element is preferably made of nonporous material.

The element to be positioned in the tissue or vessel and possibly the reinforcement element is/are preferably made of flexible material. This makes the introduction of the device into the tissue or vessel easier and reduces pain and unpleasantness during wearing thereof.

According to an alternative embodiment, the element is formed by a tube made of the porous material, an inlet tube made of nonporous material being positioned in the cavity of the tube, a connection element for connecting the inlet line for the connection to the container for the substance to be delivered being positioned at one end of the tube, this substance being conveyed via the inlet tube to the at least one inlet located at the free end of the tube, and the at least one outlet for the connection to the drain line for removal of the bodily substances from the tissue or vessel extracted via the porous material of the tube being positioned on the connection element. On the side of the device on which the connection of the drain line for draining the bodily substances is positioned, the connection for connecting to the inlet line for supplying the substance to be delivered is connected to the end of the nonporous inlet tube located there, so that if a differential pressure builds up between the inlet line and the drain line, fluids and materials dissolved therein may be conveyed from a container outside the tissue or vessel via the cavity of the inlet tube to the free end of the porous element and may be conducted from there through the porous material. While flowing through the porous material along the longitudinal axis of the element, substances may pass from the porous material into the tissue and substances may also pass from the tissue into the porous material. After traversing the porous element, fluids and bodily materials dissolved therein are suctioned off from the end of the porous element and transported via the drain line to a sensor or collection container.

The attachment element preferably has a hole discharging into the cavity of the inlet tube and a hole positioned on the front side of the end of the porous tube, which is connected to a hole discharging into the drain line. This represents a simple embodiment variation of the connections to the tube-shaped element.

The free end of the porous tube and possibly the nonporous inlet tube may be implemented as beveled. If stiff porous materials and/or stiff nonporous inlet tube materials are used, the device may thus also be inserted directly into the tissue or vessel.

The porous tube and possibly the nonporous inlet tube may be made of flexible material, which allows more comfortable wearing of the device.

If flexible porous materials and flexible nonporous inlet tube materials are used, the connection element may also have a hole, which corresponds to the cavity of the inlet tube, for inserting a needle. A needle inserted via the hole into the cavity of the inlet tube allows easier placement of the device in the tissue or vessel.

After the placement of the device, the needle is removed and the hole is preferably provided with a cover element.

It is also possible for the nonporous inlet tube to be formed by an axially displaceable hollow needle. The needle is advanced when the device is placed, so that the tip of the needle projects out of the porous tube and easier introduction of the device into the tissue or vessel is thus made possible. After the placement of the device, the needle is retracted, so that the tip of the needle is also enclosed by the porous tube and therefore more comfortable wearing of the device is made possible.

The inlet line, drain line, and the porous element and possibly the connection element and the inlet tube may be glued, welded, or press fit with one another. Arbitrary combinations of these connection techniques are possible.

The inlet line, drain line, possibly the connection element, and the inlet tube may also be integrally formed with the porous element.

A system may be provided for delivery of substances to a tissue or vessel depending on the concentration of a bodily substance in the tissue or vessel having a device described above, at least one container for the substance to be delivered, at least one pump for draining the extracted bodily substance, a sensor for measuring the concentration of the bodily substance, a unit for calibrating the measurement of the concentration of the bodily substance, a unit for regulating the quantity of the substance to be delivered depending on the measured concentrations of the bodily substance, and a collection container for the extracted substances. The delivery of insulin to subcutaneous tissue depending on the measured tissue glucose concentration is thus possible in particular.

To provide a compact unit, the at least one container for the substance to be delivered, the at least one pump, the sensors, the calibration unit, the regulatory unit, and the collection container are preferably positioned in a shared housing.

The unit for calibrating the measurement of the tissue concentration of the bodily substances is advantageously formed by a unit for measuring at least one value of at least one marker parameter of the extracted tissue fluid. Particularly, such a marker parameter may be the conductivity of the extracted tissue fluid In this regard, explicit reference is made to the disclosure with respect to calibration using endogenous and/or exogenous marker parameters of WO 88/05643.

According to a further feature of the invention, the sensor for measuring the concentration of the bodily substance and the calibration unit are integrated in the device. Using such an arrangement, one fluid line may be dispensed with and may be replaced by a line having a comparatively rapid signal transmission.

It may also be achieved by an above-mentioned method, bodily substances being removed at the location of the tissue or vessel at which the substance is delivered and being supplied to a sensor for measuring the concentration of the bodily substance using the same element. Because at least one substance is introduced into the tissue or vessel and also at least one bodily substance is removed from the tissue or blood to measure its concentration using one single element, a lower stress of the patient results, since only one element must be introduced into the tissue or vessel.

For example, for the use in type 1 diabetes, insulin is delivered to the subcutaneous tissue and, at the same location of the insulin delivery, tissue fluid is removed to measure the tissue glucose concentration and the dose of the insulin to be delivered is regulated in accordance with the measured tissue glucose concentration. A so-called closed loop system is thus provided, which simulates the glucose-regulating function of a healthy pancreas. The measurement of the tissue glucose concentration is preferred to the measurement of the blood glucose concentration because of the risks in connection with continuous blood glucose measurement, such as infections, hemorrhages, and blood clotting. The tissue glucose concentration correlates very well with the blood glucose concentration.

Tissue fluid is advantageously removed continuously and therefore, for example, tissue glucose concentration is ascertained continuously, through which both timely detection of small blood glucose setpoint value deviations and also a timely correction of these deviations are made possible by adapting the insulin supply, so that pronounced hypoglycemia and hyperglycemia may be effectively avoided during treatment.

The measurement of the tissue glucose concentration is preferably calibrated permanently to be able to make a reliable statement about the actual blood glucose concentration.

The present invention will be explained in greater detail on the basis of the attached drawing.

### Therein,

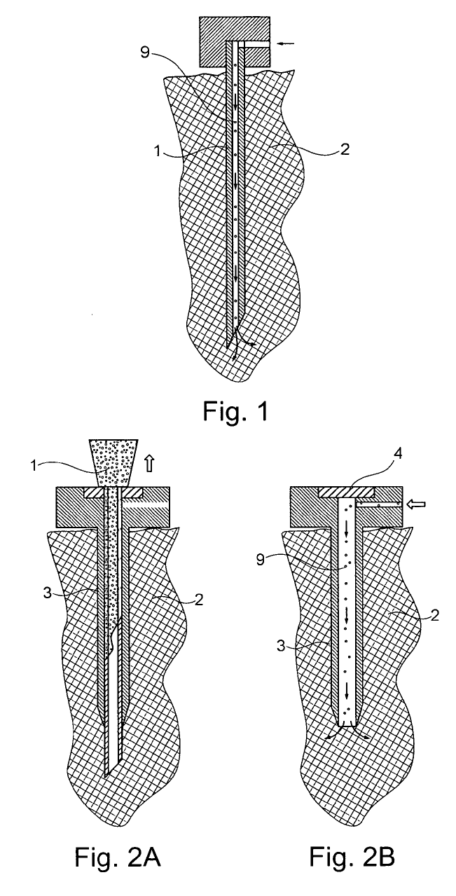

FIG. 1 shows a schematic sectional diagram of a stiff, hollow transcutaneous needle for the subcutaneous delivery of a substance using a pump;

FIGS. 2A and 2B show schematic sectional images of a flexible, transcutaneous cannula for the subcutaneous administration of a substance using a pump;

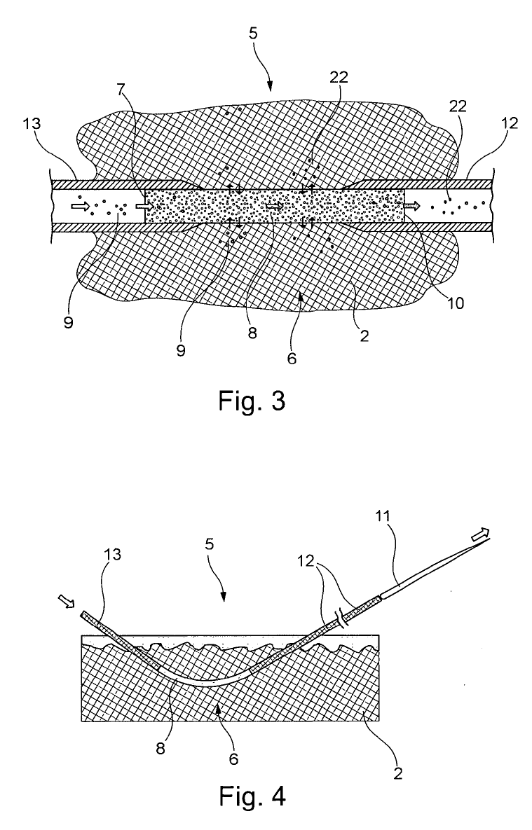

FIG. 3 shows an embodiment of the device according to the invention in a sectional image;

FIG. 4 shows the device shown in FIG. 3 during the procedure of placement in the subcutaneous tissue;

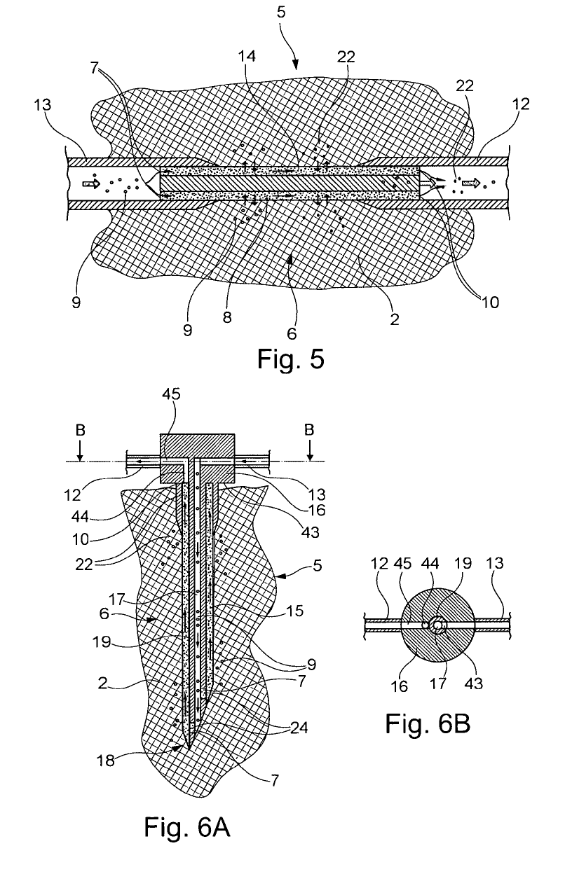

FIG. 5 shows a further embodiment of the device having a reinforcement element;

FIG. 6A shows a further embodiment of a device having a tubular porous element;

FIG. 6B shows a section through the device along the section line B-B in FIG. 6A;

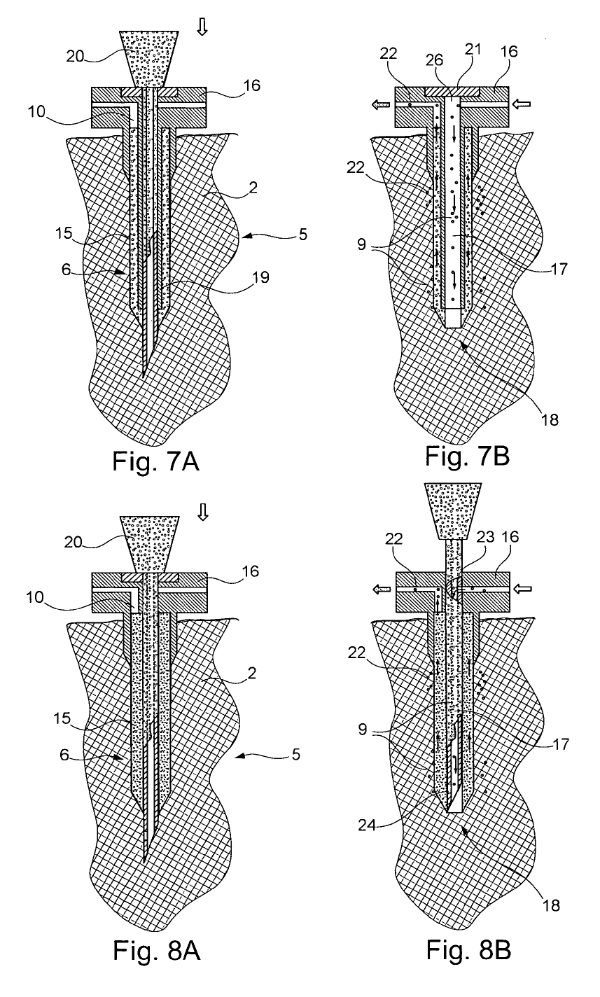

FIGS. 7A and 7B show a further embodiment of the present invention;

FIGS. 8A and 8B show a further embodiment of the device;

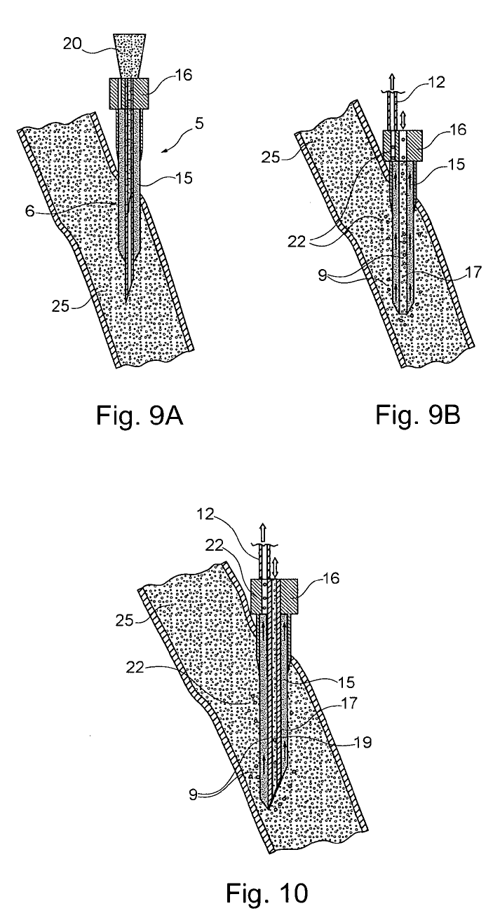

FIGS. 9A and 9B show a use of the device shown in FIGS. 8A and 8B in a vessel;

FIG. 10 shows the use of the embodiment of the device shown in FIG. 6 in a vessel;

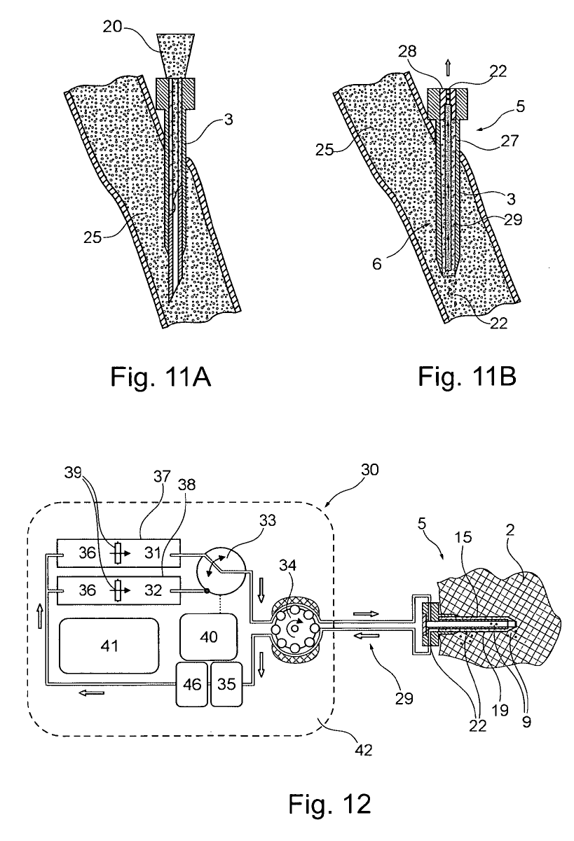

FIGS. 11A and 11B show a further embodiment of the device according to the invention during use in a vessel;

FIG. 12 shows a block diagram of an apparatus for the simultaneous delivery of a substance to the subcutaneous tissue and extraction of bodily substances from the subcutaneous tissue using a device according to the invention; and

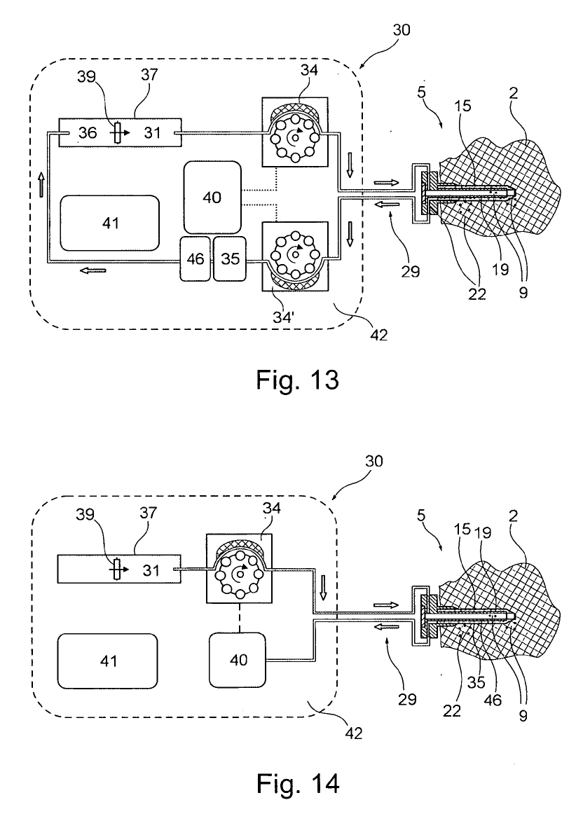

FIG. 13 and FIG. 14 show two further embodiments of an apparatus for the simultaneous delivery of a substance to the subcutaneous tissue and extraction of bodily substances from the subcutaneous tissue using a device according to the invention.

FIG. 1 shows a sectional image of a transcutaneous needle 1 made of metal as is used for the delivery of substances 9, such as insulin, with the aid of a pump (not shown). The needle 1 is inserted by the patient himself into the subcutaneous tissue 2 and connected to a line for supplying the substance 9 from a container (not shown). The substance 9 and/or the insulin is delivered through the hollow needle 1 into the subcutaneous tissue 2. Transcutaneous needles 1 of this type are unsuitable for the removal of bodily substances from the subcutaneous tissue 2.

FIGS. 2A and 2B show a flexible, transcutaneous cannula 3 as is used, for example, in the therapy of diabetes with the aid of insulin pumps. The cannula 3 made of elastic material is inserted by the patient himself into the subcutaneous tissue 2 with the aid of a hollow needle 1 made of metal. After the insertion, the hollow needle 1 is removed as shown in FIG. 2B and the opening is closed by a cover element 4. After connecting the cannula 3 to a corresponding line (not shown) for supplying the substance 9 to be delivered, such as insulin, the delivery of the substance 9 to the subcutaneous tissue 2 may be performed. Cannulas 3 of this type offer increased wearing comfort due to the elevated elasticity of the materials used. However, transcutaneous cannulas 3 of this type are also unsuitable for removing bodily substances from the subcutaneous tissue 2.

FIG. 3 shows a first embodiment of the present invention having a device 5 for delivery and removal of substances 9 and 22, respectively, using an element 6 to be positioned in the tissue 2 having an inlet 7 for supplying the substances 9 to be delivered and an outlet 10 for draining the bodily substances 22 removed. The inlet 7 is connected via an inlet line 13 to a container (not shown) for the substance 9 to be delivered and/or multiple containers for the substances 9 to be delivered. A corresponding drain line 12 is positioned at the outlet 10, via which the bodily substances 22 and possibly undelivered substances 9 may be drained. In the exemplary embodiment illustrated, the element 6 comprises a rod 8 made of porous material. Through a pressure differential built up between the lines 13, 12 connected to the inlet 7 and outlet 10, the substances 9 to be delivered are introduced via the inlet 7 into the end of the rod 8 made of porous material and conducted through the pores of the porous material in the direction of the opposite end of the rod 8. Because of concentration differences between the substances in the porous material of the rod 8 and the substances in the tissue 2 and/or because of pressure differences between porous material and tissue 2, substances 9 may be conveyed from the porous material of the rod 8 into the tissue 2 and/or bodily substances 22 may be conveyed from the tissue 2 into the porous material of the rod 8 while flowing through the porous material. After flowing through the porous rod 8, the extracted bodily substances 22 and possibly the undelivered residue of the substances 9 are drained via the drain line 12 connected to the outlet 10. The device 5 is placed at a suitable location of the tissue 2 and thus allows both the delivery of a substance 9 to the tissue 2 and also the extraction of bodily substances 22 from the tissue fluid of the tissue 2. This embodiment variation is especially suitable for use in the subcutaneous tissue 2. A use of the device 5 in other tissue parts of the human or animal body and in vessels, particularly blood vessels, is also possible.

FIG. 4 shows the device 5 according to FIG. 3 during the introduction into the subcutaneous tissue 2. In this case, the rod 8, which is preferably made of flexible porous material, is introduced into the subcutaneous tissue 2 with the aid of the needle 11 which is mounted on a drain line 12 connected to the outlet 10. The inlet line 13 and drain line 12 may be glued, welded, press fit or integrally formed with the element 6 and/or rod 8. The inlet line 13 may also be plugged onto the inlet 7 and the drain line 12 may be plugged onto the outlet 10. The pressure difference between the inlet line 13 connected to the inlet 7 and the drain line 12 connected to the outlet 10 may be built up to convey the substances 9, 22 in the lines 13, 12 and in the porous rod 8 by a corresponding pump system (not shown) in the form of a positive pressure (so-called push mode), a negative pressure (so-called pull mode), or both a positive and also a negative pressure (push-pull mode). The device 5 according to FIGS. 3 and 4 is characterized by a simple construction and may therefore be produced cost-effectively. During placement of the device 5 in the tissue 2 or vessel, the skin has to be tunneled under, however, and therefore the skin has to be penetrated twice, which may cause less wearing comfort and increased risk of infection. The cost-effective embodiment variation illustrated in FIGS. 3 and 4 will therefore be well suitable for those applications in which only relatively short wearing times of the device 5 are necessary.

FIG. 5 shows a further embodiment of the present invention, a reinforcement element 14 made of nonporous material being positioned in the element 6 to be positioned subcutaneously. The reinforcement element 14 is preferably produced from the same material as the element 6, only without pores. The element 6 made of porous material may, for example, be produced by sintering plastic, ceramic, or metal. In this case, areas without porosity may also be produced in the element 6, so that it is also conceivable to integrate the reinforcement element 14 in the element 6 and produce them in one procedure. In addition, the porosity may vary depending on the location of the element 6 and, for example, a porosity gradient may be produced which supports the delivery of the substances 9 and the extraction of the bodily substances 22.

FIGS. 6A and 6B show a further embodiment of the invention, the element 6 to be positioned in the subcutaneous tissue 2 being formed by a tube 15 made of porous material. The outlet 10 for connection to the drain line 12 for draining the bodily substances 22 extracted via the porous material of the tube 15 is positioned at one end of the porous tube 15. In addition, an inlet tube 19 made of nonporous material is fitted in the cavity of the porous tube 15. On the side of the device 5 on which the outlet 10 for the connection to the drain line 12 for draining the bodily substances 22 is positioned, an attachment element 16 for connection to the inlet line 13 for supplying the substances 9 to be delivered is attached at the end of the nonporous inlet tube 19 located there. The attachment element 16 comprises, as shown in FIG. 6B, a hole 43 discharging into the cavity 17 of the inlet tube 19 and a hole 44 positioned at the front end of the end of the porous tube 15, which is connected to a hole 45 discharging into the drain line 12. Upon buildup of a differential pressure between the lines 13, 12 connected via the attachment element 16 and outlet 10, as shown in FIG. 6A, the substances 9 to be delivered may be conveyed via the cavity 17 of the inlet tube 19 to the free end 18 of the inlet tube 19 and porous tube 15 and conducted from there via the inlets 7 into the porous material of the tube 15. While flowing through the porous material along the longitudinal axis of the tube 15, the substances 9 to be delivered may pass from the porous material into the subcutaneous tissue 2 and also the substances 22 may pass from the subcutaneous tissue 2 into the porous material. After flowing through the porous material along the longitudinal axis of the tube 15, the extracted bodily substances 22 and possibly the not delivered residue of the substances 9 are drained via the drain line 12 attached to the outlet 10 at the end of the tube 15. The flow of the substances 9 to be delivered and the extracted bodily substances 22 is illustrated on the basis of the arrows in FIG. 6A. The tube 15 and/or the inlet tube 19 are preferably made of stiff materials and the free ends 18 of the porous tube 15 and nonporous inlet tube 19 are implemented as beveled, so that the device 5 may be penetrated into the subcutaneous tissue 2 without further aids. Comparably to the stiff transcutaneous needle 1 shown in FIG. 1 for subcutaneous delivery of insulin using a pump according to the prior art, the embodiment variation shown in FIGS. 6A and 6B may be introduced by the patient himself into the subcutaneous tissue 2 and used for delivery of a substance 9, such as insulin. An essential advantage of this embodiment variation in relation to the transcutaneous needle 1 according to the prior art is the additional possibility of the removal of bodily substances 22, such as glucose, for the purpose of measuring the tissue glucose concentration. Because of the rigidity of the materials used, however, the embodiment variation according to FIG. 6 has a restricted wearing comfort comparable to the transcutaneous needle 1 shown in FIG. 1.

FIGS. 7A and 7B show a further embodiment of the device 5 according to the invention, the porous tube 15 and the nonporous inlet tube 19 preferably being made of flexible material, which allows more comfortable wearing of the device 5. In order to make the placement of the device 5 in the subcutaneous tissue 2 easier when flexible materials are used, a stiff needle 20 is inserted into the cavity 17 of the inlet tube 19 via a hole 26, and the device 5 is inserted into the subcutaneous tissue 2 with the aid of the needle 20. After the insertion of the device 5 into the subcutaneous tissue 2, the needle 20 is removed in accordance with FIG. 7B and the hole 26 is closed by a cover element 21. Comparably to the flexible transcutaneous cannula 3 for subcutaneous delivery of substances using a pump according to the prior art shown in FIG. 2, the embodiment variation in accordance with FIGS. 7A and 7B may be introduced by the patient himself into the subcutaneous tissue 2 and used for delivery of substances 9. An essential advantage of this embodiment variation in relation to the transcutaneous cannula according to the prior art is, however, the additional possibility of removing bodily substances 22. Therefore, the device 5 is suitable, for example, for use for the therapy of diabetes, since insulin or another glucose-regulating medication may be delivered to the subcutaneous tissue 2 and the glucose concentration in the tissue fluid may also be ascertained. Experiments have shown optimum results when the insulin delivery and the measurement of the tissue glucose concentration occur at essentially the same location in the subcutaneous adipose tissue. The relation between blood glucose concentration and glucose concentration in adipose tissue was found to be stable despite the extremely high insulin concentrations present at the delivery site. A use of the device 5 in other tissue parts of the human or animal body and in vessels is also possible.

FIGS. 8A and 8B show a further embodiment of a device 5, the nonporous inlet tube 19 being implemented as a needle 20 movable in the porous tube 15, in contrast to the variation shown in FIG. 6. The needle 20 is positioned in accordance with FIG. 8A for insertion, so that its tip projects beyond the tube 15 made of porous material and easier introduction of the device 5 into the subcutaneous tissue 2 is thus made possible. After the introduction of the device 5, as shown in FIG. 8B, the needle 20 is retracted somewhat, so that the tip of the needle 20 is also enclosed by the porous tube 15 and therefore more comfortable wearing of the device 5 is made possible. In this needle position, the supply line for the substances 9 in the attachment 16 also corresponds to a hole 23 in the needle 20, so that the substances 9 may be conveyed via the cavity 17 of the needle 20 to the free end 18 of the porous tube 15. At least one hole 24 may be positioned at the tip of the needle 20, which makes the redirection of the substances 9 into the porous material of the tube 15 easier.

FIGS. 9A and 9B show the device 5 according to FIG. 8A during use in a vessel 25, such as a blood vessel, wherein according to FIG. 9A, the needle 20 being positioned in such a way that its tip projects beyond the tube 15 made of porous material and the insertion into the vessel 25 is thus made easier. After the insertion into the vessel 25, however, in contrast to the variation shown in FIG. 8B, the needle 20 is removed and a substance 9 is introduced into the vessel 25 via the cavity 17 of the porous tube 15. Blood may also be suctioned out of the vessel 25 via the cavity 17, for example. Substances 22 of the blood may be drained via a drain line 12 in the attachment element 16 connected to the tube 15 made of porous material and supplied to an analysis, for example. If the pore size of the porous material of the tube 15 is selected so that blood cells (e.g., erythrocytes) may not penetrate into the porous material, for example, blood plasma may be suctioned off via the drain line 12.

FIG. 10 shows the device 5 in accordance with FIG. 6 during use in a vessel 25, a substance 9 being able to be introduced into the vessel 25 via the cavity 17 of the inlet tube 19 according to FIG. 10. Blood may also be suctioned out of the vessel 25 via the cavity 17. If the pore size of the porous material of the tube 15 is selected so that blood cells (e.g., erythrocytes) may not penetrate into the pores of the tube 15, blood plasma may be suctioned off via the drain line 12 connected to the attachment element 16, for example.

FIGS. 11A and 11B show a variant of a device 5 which is suitable for delivery of substances 9 to vessels 25 and for removal of blood plasma from the same vessels 25. Similarly to the embodiment according to FIG. 2A, a cannula 3 made of nonporous material is introduced into the vessel 25 with the aid of a needle 20. After the placement of the cannula 3, the needle 20 is removed and, corresponding to FIG. 11B, an element 27 made of porous material is introduced into the cannula 3 and fixed using an attachment element 28. In this case, the dimension of the element 27 is selected in such a way that a gap 29 remains free between the inner wall of the cannula 3 and the element 27 made of porous material, into which the blood may penetrate. The surface of the element 27 made of porous material, via which the bodily substances 22 of the blood to be analyzed are extracted, thus becomes larger and the removal efficiency is therefore increased. If the pore size of the porous material of the element 27 is selected so that blood cells may not penetrate into the porous material, blood plasma may be removed via the element 27, for example.

FIG. 12 shows a use of the device 5 according to the invention for delivery of substances 9, such as insulin, to the subcutaneous tissue 2 and for extraction of bodily substances 22, such as tissue glucose, from the subcutaneous tissue 2. The device 5 is penetrated and fixed in the subcutaneous tissue 2 and connected to the device 30 via lines 29. Containers 31, 32, which contain the substance 9 to be delivered to the subcutaneous tissue 2, are located in the housing 42 of the device 30. The containers 31, 32 preferably contain the substance 9 in different concentrations. The substance from the container 31 or 32, conveyed by a pump 34, reaches the device 5 via a changeover switch 33 and is at least partially delivered to the subcutaneous tissue 2. Simultaneously, bodily substances 22 of the subcutaneous tissue 2, such as the glucose concentration, are extracted via the tube 15 made of porous material and, also conveyed by the pump 34, supplied to a sensor 35 for measuring the tissue concentration of the bodily substances. After the analysis, the tissue fluid reaches the collection container 36. The containers 31 and 36 and/or 32 and 36 may be formed by a shared container 37 and/or 38, a movable wall 39 being able to be provided for separating the substance 9 in the container 31 and/or 32 and the collected cleaning or tissue fluid in the container 36. A regulatory unit 40 receives the values measured by the sensor 35, such as a glucose sensor, and regulates the quantity of the substance 9 and/or the insulin to be delivered through corresponding switching of the changeover switch 33. A unit 46 for calibrating the measurement of the tissue concentration of the bodily substances 22 is advantageously also provided, which may be formed, for example, by a unit for measuring the conductivity of the collected tissue fluid. Furthermore, a unit 41 for supplying electrical power is provided, which is preferably formed by rechargeable accumulators. The housing 42 of the device 30 may be sealed and may be made of biocompatible material, such as titanium, to allow implantation.

FIG. 13 shows a further variant of the use of the device 5 according to the invention for delivery of a substance 9, such as insulin, to the subcutaneous tissue 2 and for extracting bodily substances 22, such as tissue glucose, from the subcutaneous tissue 2. In contrast to the variant shown in FIG. 12, the embodiment variation shown in FIG. 13 has two pumps 34, 34′ and only one container 31 for the substance 9 to be delivered to the subcutaneous tissue 2. The pumps 34, 34′ are provided for the separate conveyance of the substance 9 to be delivered from the container 31 to the device 5 and for conveying the bodily substance 22 from the device 5 to the sensor 35. The (rotational) speed of the pump 34 for conveying the substance 9 to be delivered is regulated in accordance with the measured tissue concentrations of the bodily substances 22. The (rotational) speed of the pump 34′ for conveying the bodily substances 22 may also be regulated by the regulatory unit 40. Using this arrangement, a very high efficiency in the delivery of the substance 9 may be achieved.

Finally, FIG. 14 shows a further variant of the use of the device 5 according to the invention for delivery of a substance 9, such as insulin, to the subcutaneous tissue 2 and for measuring bodily substances 22, such as tissue glucose, in the subcutaneous tissue 2. In contrast to the variant shown in FIG. 13, in the embodiment variant according to FIG. 14, the sensor 35 and possibly the calibration unit 46 are integrated in the device 5. The sensor 35 and possibly the calibration unit 46 are preferably attached in the wall of the porous tube 15. The signals from the sensor 35 and possibly the calibration unit 46 are transmitted via a line 44 to the regulatory unit 40. In this case, the transmission of the signals may be performed electrically or optically. Using this arrangement according to FIG. 14, because a fluid line between device 5 and device 30 is dispensed with and this fluid line is replaced by a line having comparatively rapid signal transmission, a very short response time of the device 30 and therefore a rapid and robust regulation of the concentration of the bodily substances 22, such as the glucose concentration, may be achieved.

Notwithstanding the embodiment of the element 6 to be positioned in the subcutaneous tissue in the form of a rod 8 or tube 15, other variants, for example, in the form of a disk, are also conceivable. The shape of the element 6 made of porous material is not of decisive significance. It is essential that the substances 9 to be delivered are brought to the end of the inlet line 13 and conducted from there through the porous element 6 in the direction of the opposing beginning of the drain line 12, so that during the traversal of the porous element 6 the substances 9 may be delivered to the tissue 2 or vessel 25 and bodily substances 22 may be extracted from the tissue 2 or vessel 25 via the mantle surface of the porous element 6. The extracted bodily substances 22 may then be drained via the drain line 12 from the beginning of the drain line 12.

It should be noted that the term “comprising” does not exclude other elements or features and the “a” or “an” does not exclude a plurality. Also elements described in association with different embodiments may be combined.

It should also be noted that reference signs in the claims shall not be construed as limiting the scope of the claims.

1. A device for delivery of substances to and removal of bodily substances from a tissue or vessel of a body, comprising an element to be positioned in the tissue or vessel, characterized in that the element is at least partially made of porous material, and comprises at least one inlet for supplying the substances to be delivered and at least one outlet for draining the bodily substances, the inlet being connectable via an inlet line to a container for the substances to be delivered, so that in the event of a pressure gradient between inlet and outlet, during the traversal of the porous element, the substance to be delivered is delivered to the tissue or vessel via the mantle surface of the porous element and the bodily substances are extracted from the tissue or vessel via the mantle surface of the porous element and drained via a drain line connectable to the outlet.

2. The device according to claim 1, characterized in that the element is at least partially made of porous metal.

3. The device according to claim 1, characterized in that the element is at least partially made of porous ceramic material.

4. The device according to claim 1, characterized in that the element is at least partially made of porous plastic.

5. The device according to claim 1, characterized in that the size of the pores of the porous material is 0.02 to 200 μm.

6. The device according to claim 1, characterized in that the element has pores of different sizes.

7. The device according to claim 6, characterized in that the element has a porosity gradient.

8. The device according to claim 1, characterized in that the element is at least partially made of hydrophobic porous material.

9. The device according to claim 1, characterized in that the element is at least partially made of hydrophilic porous material.

10. The device according to claim 1, characterized in that the element is formed by a rod made of the porous material, the inlet for attaching the inlet line for the connection to the container for the substance to be delivered being positioned at one end of the rod and the outlet for attaching the drain line for draining the extracted bodily substances being positioned at the other end of the rod, so that the delivery of the substance to the tissue or in the vessel and the extraction of the bodily substances from the tissue or vessel occurs via the mantle surface of the rod.

11. The device according to claim 10, characterized in that a reinforcement element is positioned in the interior of the rod.

12. The device according to claim 11, characterized in that the reinforcement element is made of nonporous material.

13. The device according to claim 10, characterized in that the element and possibly the reinforcement element are made of flexible material.

14. The device according to claim 1, characterized in that the element is formed by a tube made of the porous material, an inlet tube made of nonporous material being positioned in the cavity of the tube made of the porous material, wherein at one end of the tube an attachment element is positioned for attaching the inlet line for connection to the container for the substance to be delivered, said substance being conveyed via the inlet tube to the at least one inlet located at the free end of the tube, and the at least one outlet for the attachment to the drain line for draining the bodily substances from the tissue or vessel extracted via the porous material of the tube made of the porous material being positioned on the attachment element.

15. The device according to claim 14, characterized in that the attachment element has a hole discharging into the cavity of the inlet tube and a hole positioned on the front side of the end of the tube made of the porous material, which is connected to a hole discharging into the drain line.

16. The device according to claim 14, characterized in that the free end of the porous tube and possibly of the inlet tube is implemented as bevelled.

17. The device according to claim 14, characterized in that the tube made of the porous material and possibly the inlet tube is made of flexible material.

18. The device according to claim 15, characterized in that the attachment element comprises a hole for inserting a needle, which hole corresponds to the cavity of the inlet tube.

19. The device according to claim 18, characterized in that the hole is provided with a cover element.

20. The device according to claim 14, characterized in that the inlet tube is formed by an axially displaceable needle.

21. The device according to claim 10, characterized in that the inlet line, the drain line, and the porous element, and possibly the attachment element and the inlet tube, are glued to one another.

22. The device according to claim 10, characterized in that the inlet line, the drain line, and the porous element, and possibly the attachment element and the inlet tube, are welded to one another.

23. The device according to claim 10, characterized in that the inlet line, the drain line, and the porous element, and possibly the attachment element and the inlet tube, are press fit with one another.

24. The device according to claim 10, characterized in that the inlet line, the drain line, and possibly the attachment element and the inlet tube, are integrally formed with the porous element.

25. A system for delivery of substances to a tissue or vessel depending on the concentrations of a bodily substance in the tissue or vessel, comprising a device according to claim 1, at least one container for the substance to be delivered, at least one pump for draining the extracted bodily substance, a sensor for measuring the concentration of the bodily substance, a unit for calibrating the measurement of the concentration of the bodily substance a unit for regulating the quantity of the substance to be delivered depending on the measured concentrations of the bodily substance, and a collection container for the extracted substances.

26. The system according to claim 25, characterized in that the at least one container for the substance to be delivered, the at least one pump, the regulatory unit, the collection container, and possibly the sensor and the calibration unit are positioned in a shared housing.

27. The system according to claim 25, characterized in that the calibration unit is formed by a unit for measuring the conductivity of the collected tissue fluid.

28. The system according to claim 25, characterized in that the sensor for measuring the concentration of the bodily substance and the calibration unit is integrated into the device.

29. A method for delivery of substances to a tissue or vessel, the substances being delivered in predefined doses via an element which is introducible into the tissue or vessel, characterized in that, using the same element, bodily substances are removed at the location of the tissue or vessel at which the substance is delivered and are supplied to a sensor for measuring the concentration of the bodily substance.

30. The method according to claim 29, characterized in that insulin is delivered and tissue fluid is removed at the same location of the insulin delivery for measuring the tissue glucose concentration and the dose of the insulin to be delivered is regulated in accordance with the measured tissue glucose concentration.

31. The method according to claim 29, characterized in that tissue fluid is removed continuously.

32. The method according to claim 29, characterized in that the measurement of the tissue glucose concentration is preferably calibrated permanently.

33. The method according to claim 29, characterized in that the element is at least partially made of porous material.
